(19) United States
(12) Patent Application Publication
(10) Pub. No.: US 2007/0274440 A1
(43) Pub. Date: Nov. 29, 2007

(54) AUTOMATIC DETERMINATION OF CEPHALOMETRIC POINTS IN A THREE-DIMENSIONAL IMAGE

(76) Inventors: David Phillipe Sarment, Ann Arbor, MI (US); Joseph Webster Stayman, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/747,487

(22) Filed: May 11, 2007

Related U.S. Application Data
(60) Provisional application No. 60/799,588, filed on May 11, 2006.

(51) Int. Cl. A61B 6/14

(52) U.S. Cl. 378/38

(57) ABSTRACT

A CT scanner generates a three-dimensional CT image that is used to construct a ceph image. The computer automatically outlines various parts of the patient to automatically locate points and/or contours that are displayed on the three-dimensional image. The computer also automatically calculates a plurality of cephalometric points that are displayed on the three-dimensional CT image. Once the contours and the ceph points located, the computer determines angles between certain ceph points and/or the contours and compares the angles to stored standard angles. This provides an objective standard for assessing the appearance of the patient and can be used as a guideline in planning any procedure that may affect the appearance of the patient.

### REFERENCE TO RELATED APPLICATIONS

This application claims priority to U.S. Provisional Patent Application No. 60/799,588 filed May 11, 2006.

### BACKGROUND OF THE INVENTION

The present invention relates generally to a CT scanner system for generating and analyzing three-dimensional cephalometric scans used by orthodontists and other doctors.

Maxillofacial surgeons, orthodontists and other doctors use cephalometrics to diagnose, plan and predict maxillofacial surgeries, orthodontic treatments and other treatments that could affect the shape and appearance of a face of a patient. One important part of the cephalometric (“ceph”) analysis is starting with ceph images of the patient's head. Primarily, two-dimensional lateral x-ray ceph images are taken of the patient's head, although other additional images can be used.

Once the ceph image has been obtained, the doctor must manually outline the contours on the ceph image and manually locate and mark defined “ceph points” on the ceph image. Based upon the arrangement of the ceph points, and based upon a comparison to one or more standards, a doctor can make an objective goal for the patient's appearance after the surgery or treatment.

It is time consuming for the doctor to outline the contours and perform the analysis to determine the ceph points. Software is available to assist the doctor in plotting the ceph points on the ceph image using a computer mouse. The software also assists in performing a comparison between the ceph points and stored standards. However, locating and marking the ceph points on the ceph image is tedious and time-consuming.

Software has also been used to automatically identify the ceph points in a two-dimensional image. However, locating and marking the ceph points in two dimensions is difficult as the patient's head is three-dimensional.

### SUMMARY OF THE INVENTION

A CT scanner includes a gantry that supports an x-ray source and a complementary flat-panel detector spaced apart from the x-ray source. The x-ray source generates x-rays that are directed toward the detector to create an image. As the gantry rotates about the patient, the detector takes a plurality of x-ray images at a plurality of rotational positions. The CT scanner further includes a computer that generates and stores a three-dimensional CT image created from the plurality of x-ray images.

The three-dimensional CT image is used to construct a ceph image of the patient. The computer automatically outlines various parts of the patient to automatically locate points and/or contours that are displayed on the three-dimensional image. The computer also automatically calculates a plurality of cephalometric points that are displayed on the three-dimensional CT image.

The doctor can review the contours and the ceph points shown on the three-dimensional CT image. The doctor can edit and move the ceph points to a desired location to the extent the doctor does not agree with the automatic determination of the location of the ceph points.

Once the contours and the ceph points are located on the three-dimensional image, the computer determines angles between certain ceph points and/or the contours and compares the angles to stored standard angles. This provides an objective standard for assessing the appearance of the patient and can be used as a guideline in planning any procedure that may affect the appearance of the patient.

### BRIEF DESCRIPTION OF DRAWINGS

FIG. 1 illustrates a first embodiment CT scanner;

FIG. 2 illustrates a second embodiment CT scanner;

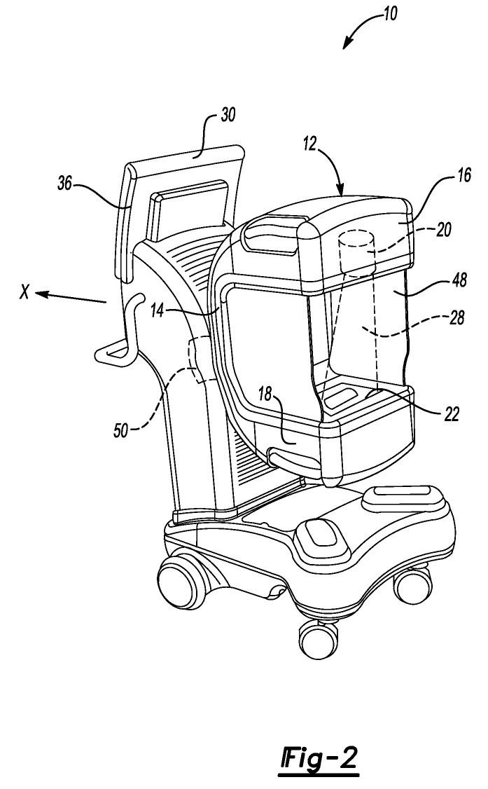

FIG. 3 illustrates a computer employed with the CT scanner; and

FIG. 4 illustrates a view of a three-dimensional image of a patient showing contours and ceph points.

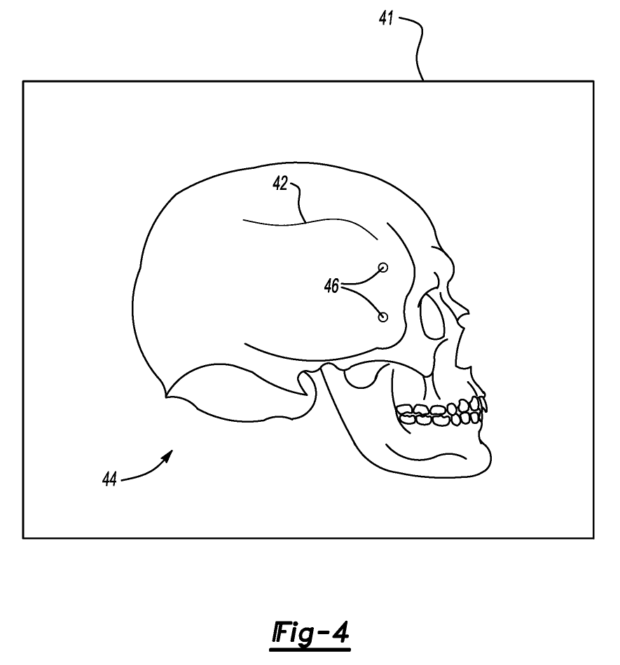

### DETAILED DESCRIPTION OF THE PREFERRED EMBODIMENTS

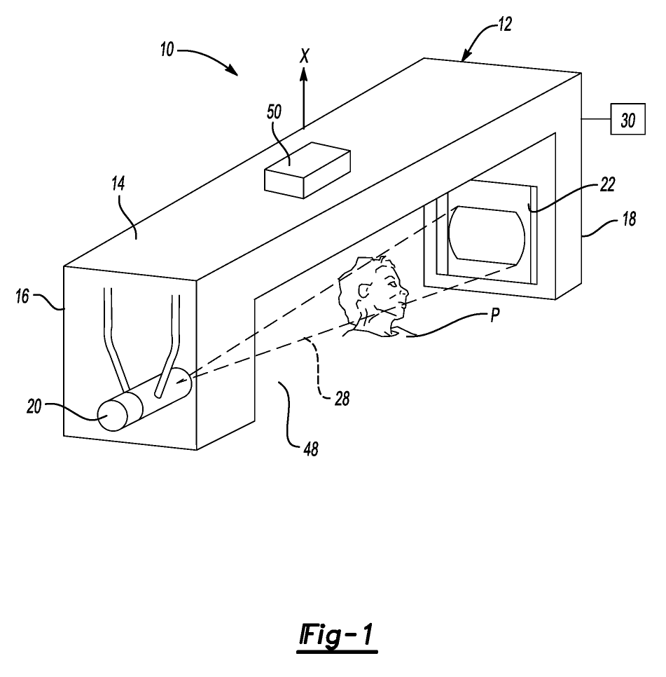

FIG. 1 illustrates a CT scanner 10 of including a gantry 12 that supports and houses components of the CT scanner 10. Suitable CT scanners 10 are known. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22 spaced apart from the x-ray source 20. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

A part of the patient P, such as a head, is received in a space 48 between the first arm 16 and the second arm 18. A motor 50 rotates the gantry 12 about an axis of rotation X to obtain a plurality of x-ray images of the patient P at the plurality of rotational positions. The axis of rotation X is positioned between the x-ray source 20 and the detector 22. The gantry 12 can be rotated approximately slightly more than 360 degrees about the axis of rotation X. In one example, as shown in FIG. 1, the axis of rotation X is substantially vertical. Typically, in this example, the patient P is sitting upright. In another example, the axis of rotation X is substantially vertical, and the patient P is typically lying down on a table 70.

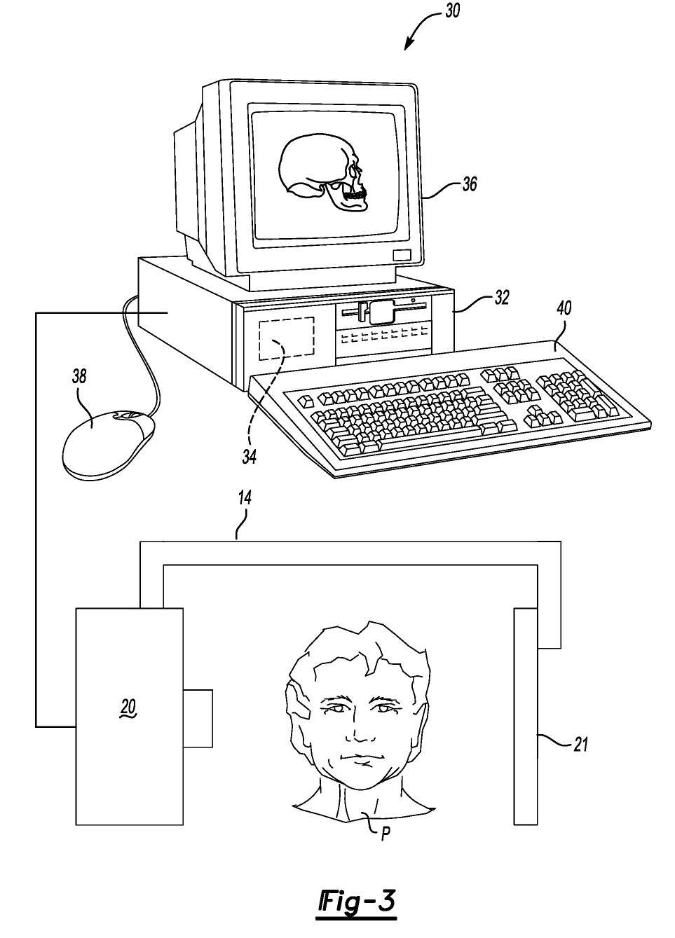

As shown schematically in FIG. 3, the CT scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30. The computer 30 generates a three-dimensional CT image from the plurality of x-ray images utilizing any known techniques and algorithms. The three-dimensional CT image is stored on the storage 34 of the computer 30 and can be displayed on the display 36 for viewing.

In operation, the part of the patient P to be scanned is positioned between the first arm 16 and the second arm 18 of the gantry 12. In one example, the part of the patient P is the patient's P head. The x-ray source 20 generates an x-ray 28 that is directed toward the detector 22. The CPU 32 then controls the motor 50 to perform one complete revolution of the gantry 12, while the detector 22 takes a plurality of x-ray images of the head at a plurality of rotational positions. The plurality of x-ray images are sent to the computer 30. A three-dimensional CT image 41 is then constructed from the plurality of x-ray images utilizing any known techniques and algorithms. The example illustrates a three-dimensional CT image 41 constructed using the CT scanner 10 described above.

After the three-dimensional CT image 41 is constructed by the computer 30, the three-dimensional CT image 41 can be used to construct a ceph image of the patient P to be displayed on display 36. The ceph image is shown in two dimensions, although the calculations to find the ceph points 46 is done in three dimensions.

The computer 30 (or a different computer) first automatically finds the edges and outlines of the various parts of a head 44 of the patient P, such the skull, the teeth, the nose, etc. The computer 30 then automatically locates points and/or contours 42 based upon the edges of the various parts. The computer 30 may also find and outline the points and/or contours 42 based upon a relative thicknesses of the parts of the head 44 or other features that can be determined from the three-dimensional CT image 41, some of which that are not identifiable on a two-dimensional x-ray image. That is, the computer 30 identifies, outlines and stores relevant points and/or contours 42 in the three-dimensional CT image 41. The points and/or contours 42 are displayed on the three-dimensional CT image 41 on the display 36.

A plurality of ceph points 46 are localized and plotted on the three-dimensional CT image 41. The doctor can use the relationship between the points and/our contours 42 and the ceph points 46 to plan an orthodontic treatment or a surgical procedure.

The ceph points 46 are determined from a generic training set. The training set is generated using a large database of three-dimensional images. An expert panel manually locates landmarks in the three-dimensional image, and small three-dimensional cubes are formed around the landmarks. Alternatively, the spheres can be formed around the landmarks. For example, the landmark can be a tip of an incisor, a tip or base of a specific tooth or any bony landmark.

Any natural variation in the three-dimensional CT images and any variation caused by differences in the expert panel localization is accommodated for in the training set. For example, some features will not be present in all of the three-dimensional CT images (i.e., some of the patients used to form the three-dimensional CT images may be missing teeth). Additionally, there will be some variation in localization amongst the expert panel as their opinions on the locations of the specific landmarks may differ. When forming the training set, missing features (the teeth) are accommodated for by either eliminating the three-dimensional CT images of the patients that are missing teeth or by assuming that the missing feature (the teeth) does not exist, creating a “null condition.”

After the training set is defined and the landmarks are indicated, measurements are made on the training set that will be used for localization (as described below). Various types of measurements can be made on the three-dimensional cubes. For example, intensity values (i.e., the average cube), three-dimensional moments of the intensity values (mean, variance, skew, etc.), three-dimensional spatial frequency content and other decompositions of the intensity values (wavelets, blobs, etc.), including decompositions based on principal component analysis of example (typically using singular value decomposition), can be measured.

In one example, the various measurements are evaluated using cluster analysis of the training set. A good set of measurements will form separated clusters in measurement space. The degree of separation can be quantified using statistical analysis of the clusters (i.e., Gaussian assumptions and confidence intervals, etc.) to accommodate for unusually shaped clusters. For example, if there are two basic classes of a single feature, one of the classes may be a “feature cluster” which is itself composed of disconnected clusters.

After the training set is formed and the measurements are extracted, a localization search is performed. Usually, the entire three-dimensional CT image 41 is scanned and compared to the information in the training set. The three-dimensional CT image 41 and the images in the training set are similarly aligned and similarly oriented so that little image rotation is needed during scanning. Therefore, the landmarks/measurements require little translational scanning and rotation. However, there could be some automatic alignment if the images are not aligned, for example if there is any head tilt. Therefore, some measurements might require a small rotational search (i.e., over a small number of angles) which could be accommodated for by translational scanning plus a small angle search.

Every location in the three-dimensional CT image 41 is identified during localization. The selected measurements are applied to the three-dimensional CT image 41 to search for any similarity, allowing the ceph points 46 to be plotted on the three-dimensional CT image. The ceph points 46 are displayed on the display 36 for viewing by the doctor.

In a first example of localization, a matched filter/correlational approach is employed. Each anatomical feature has a mean exemplar formed from the training set. The average three-dimensional cube can be applied as a filter to the three-dimensional image in the form of a three-dimensional convolution. The resultant image provides a map of the degree of similarity to the exemplar. The peak value in the map forms the most probable location of the anatomical feature and therefore the ceph point 46. This technique can be modified to require a certain threshold that the anatomical feature is properly localized or if the feature is simply not present. This technique can also be modified to include an angular search at every position.

In another example of localization, a moments approach is employed. Each anatomical feature has a measurement vector associated with the training exemplars, e.g., the mean value of the cube, the center of mass of the cube's intensities, etc. The measurement vector is computed for every sub-cube of the patient volume. The vector is compared to the ideal feature measurement vector (based on the training data) using a vector norm to form a similarity measure. The similarity measure can be formed into a three-dimensional map for localization using the peak value as the position estimate (or applying the aforementioned “existence thresholds,” etc.) of the ceph point 46.

In a third example of localization, a local decomposition approach is employed. Each anatomical feature has a measurement vector based on its training exemplars. The measurement vectors are formed via projection of the cube onto a basis set, which may be a wavelet basis, a frequency basis, or a basis formed by principal component analysis. Every sub-cube of the patient volume is decomposed into a measurement vector based on the particular basis selection. A similarity metric is formed via a vector norm with the feature vector formed during training. A three-dimensional map is formed, and the peak similarity identifies the likely position of the anatomical feature that defines a ceph point 46.

After localization, the ceph points 46 are plotted on the display 36 relative to the points and/or contours 42. The doctor can then revise the points and/or contours 42 and the ceph points 46 illustrated on the three-dimensional CT image 41. The software program further allows the doctor to edit and move the ceph points 46 to the desired locations to the extent the doctor does not agree with the automatic determination of the location of the ceph points 46. For example, the doctor can use the mouse 38 to drag and move the ceph points 46 on the three-dimensional CT image 41 to the desired location. Even if the doctor has to modify some of the ceph points 46, the time required for performing the ceph analysis is significantly reduced.

When the ceph points 46 are finally located, the computer 30 determines angles between certain ceph points 46 and/or the points and/or contours 42 and compares those angles to stored standard angles. This provides an objective standard for assessing the appearance of the patient P and can be used as a guideline in planning any procedure that may affect the appearance of the patient P.

Three-dimensional localization has several benefits over two-dimensional localization. For one, three-dimensional structures are more unique in appearance than a two-dimensional image.

Although a preferred embodiment of this invention has been disclosed, a worker of ordinary skill in this art would recognize that certain modifications would come within the scope of this invention. For that reason, the following claims should be studied to determine the true scope and content of this invention.

What is claimed is:

1. A method of determining cephalometric points, the method comprising the steps of:
generating a three-dimensional image;
determining a plurality of contours;
displaying the plurality of contours on the three-dimensional image;
automatically calculating a plurality of cephalometric points; and
displaying the plurality of cephalometric points on the three-dimensional image.

2. The method as recited in claim 1 wherein the three-dimensional image is a three-dimensional CT image.

3. The method as recited in claim 1 wherein the steps of determining the plurality of contours and automatically calculating the plurality of cephalometric points is performed by a computer program.

4. The method as recited in claim 1 further including the steps of positioning a part of a patient between an x-ray source and an x-ray detector of a CT scanner and performing a CT scan.

5. The method as recited in claim 1 wherein the step of determining the plurality of contours includes automatically finding edges in the three-dimensional image.

6. The method as recited in claim 1 wherein the step of determining the plurality of contours is based on a relative thickness of a part in the three-dimensional image.

7. The method as recited in claim 1 further including the step of identifying, outlining and storing the plurality of contours in the three-dimensional image.

8. The method as recited in claim 1 further including the step of reviewing the plurality of contours and the plurality of cephalometric points on the three-dimensional image.

9. The method as recited in claim 8 further including the step of planning a procedure based on the step of reviewing.

10. The method as recited in claim 1 further including the step of editing the three-dimensional image by moving the plurality of cephalometric points to a desired location.

11. The method as recited claim 1 further including the step of determining an angle between certain of the plurality of cephalometric points and the plurality of contours and comparing the angle to a stored angle.

12. The method as recited in claim 1 further including the step of determining the plurality of cephalometric points.

13. The method as recited in claim 12 wherein the step of determining the plurality of cephalometric points includes the steps of obtaining generic data, measuring the generic data and plotting the generic data on the three-dimensional image based on measurements to determine the plurality of cephalometric points.

14. A method of determining cephalometric points, the method comprising the steps of:
generating a three-dimensional CT image;
determining a plurality of contours;
displaying the plurality of contours on the three-dimensional image;
automatically calculating a plurality of cephalometric points;
displaying the plurality of cephalometric points on the three-dimensional image;
reviewing the plurality of contours and the plurality of cephalometric points on the three-dimensional image; and
planning a procedure based on the step of reviewing.

15. The method as recited in claim 14 wherein the step of determining the plurality of contours and automatically calculating the plurality of cephalometric points is performed by a computer program.

16. The method as recited in claim 14 further including the step of identifying, outlining and storing the plurality of contours in the three-dimensional image.

17. The method as recited in claim 14 further including the step of editing the three-dimensional image by moving the plurality of cephalometric points to a desired location.

18. The method as recited in claim 14 further including the step of determining the plurality of cephalometric points.

19. The method as recited in claim 18 wherein the step of determining the plurality of cephalometric points includes the steps of obtaining generic data, measuring the generic data and plotting the generic data on the three-dimensional image based on measurements to determine the plurality of cephalometric points.

20. A CT scanner comprising:
an x-ray source to generate x-rays;
an x-ray detector mounted opposite the x-ray source; and
a computer that generates a three-dimensional image of a patient, wherein the computer determines a plurality of contours, displays the plurality of contours on the three-dimensional image, automatically calculates a plurality of cephalometric points and displays the plurality of cephalometric points on the three-dimensional image.

21. The CT scanner as recited in claim 20 wherein the x-ray source is a cone-beam x-ray source.

22. The CT scanner as recited in claim 20 further including a gantry including a cross-bar section, a first arm and a second arm that each extend substantially perpendicularly to the cross-bar section, wherein the x-ray source is housed in the first arm and the x-ray detector is housed in the second arm.
